(19) United States
(12) Patent Application Publication
(10) Pub. No.: US 2010/0216086 A1
(43) Pub. Date: Aug. 26, 2010

(54) Intra-oral image system having swivel head optic

(76) Inventors: John Sylvester, Stuyvesant, NY (US); Shamar Parsard, Bay Shore, NY (US); Frank Farbizio, Farmingdale, NY (US)

Correspondence Address: CLIFFORD G. FRAYNE, 136 DRUM POINT RD, SUITE 7A, BRICK, NJ, 08723, US

(21) Appl. No.: 12/657,959

(22) Filed: Feb. 1, 2010

Related U.S. Application Data
(60) Provisional application No. 61/208,349, filed on Feb. 23, 2009.

(51) Int. Cl. A61B 1/24

(52) U.S. Cl. 433/29

(57) ABSTRACT

There is disclosed an intra-oral video imaging camera for viewing and recording the interior of the oral cavity, the intra-oral video imaging camera having a rotating optic head member allowing the user to preset the camera imaging prism from a vertical, bottom dead center position in a nominal arc 160 degrees to either the left or the right of bottom dead center, thus obviating the need for the user to manipulate the user's hand and/or wrist in order to obtain the desired image.

### RELATED APPLICATIONS

Applicant claims the benefit of provisional application Ser. No. 61/208,349, filed Feb. 23, 2009.

### BACKGROUND OF THE INVENTION

1. Field of the Invention

The present invention relates to the field of video imaging, and more particularly, to an intra-oral imaging apparatus for dental imaging within the oral cavity, the imaging apparatus having a swivel optic.

2. Description of the Prior Art

Video imaging cameras or intra-oral cameras have been used for some time in the dental arts for obtaining images of the oral cavity. Initially, the intra-oral cameras were bulky and not easily maneuvered within the oral cavity in order to obtain the optimum imaging desired. Still further, early embodiments of the intra-oral camera caused discomfort to the patient as the dentist or technician attempted to maneuver the bulky camera within the oral cavity.

The bulkiness of the early cameras was caused by many factors, but one important contributing factor was the light source used to illuminate the oral cavity. Early bulky intra-oral cameras used an external light source, the light of which was transported to the optic portion of the intra-oral camera by use of fiber optics, thereby contributing to the bulk of the camera and the reduced maneuverability of the camera.

The development in use of LED light sources substantially eliminated the bulk size which was caused by the older light sources. Intra-oral cameras for viewing and imaging the oral cavity at once became much slimmer and more easily maneuverable. These slimmer video imaging assemblies reduced the discomfort to the patient and were more easily maneuverable within the oral cavity. However, because of their fixed lens, the dentist or dental technician was still required to maneuver the camera within the oral cavity by grasping the intra-oral camera with his or her fingers and turning it within the oral cavity by twisting and turning of the wrist in order to gain imaging access to all portions of the oral cavity desired.

Applicant's intra-oral camera provides for the slimmer design desired to reduce patient discomfort and to be easily grasped and maneuvered by the dentist or dental technician. Applicant's intra-oral camera further provides a swivel head optic which can be swiveled 162 degrees to the right or to the left of dead center which eliminates the need for the dentist or dental technician to go through unnecessary manipulation such as twisting and turning of the wrist in order to position the camera head at the desired location. Applicant's design allows for the dentist or dental technician to position the optic head at a select particular angle and then insert the intra-oral camera into the oral cavity to view the particular area of the oral cavity desired, the image being transported to an image processing system.

### OBJECTS OF THE INVENTION

An object of the present invention is to provide for a novel intra-oral video imaging camera incorporating a swivel optic head allowing the user to swivel the optic head to a desired angle and to then position the intra-oral camera within the oral cavity to obtain video imaging of the oral cavity.

Another object of the present invention is to provide for a novel video imaging system for intra-oral imaging in which the optic head of the video imaging unit can nominally swivel from dead center to 160 degrees to the right and/or 160 degrees to the left.

A further object of the present invention is to provide for a novel intra-oral video imaging system in which the optic head can swivel without effecting or damaging the electrical wiring harness within the intra-oral video imaging assembly.

### SUMMARY OF THE INVENTION

An intra-oral video imaging camera for viewing and recording the interior of the oral cavity, the intra-oral video imaging camera having a rotating optic head allowing the user to preset the camera imaging prism from a vertical, bottom dead center position in a nominal arc 160 degrees to either the left or the right of bottom dead center, thus obviating the need for the user to manipulate the user's hand and/or wrist in order to obtain the desired image.

### BRIEF DESCRIPTION OF THE DRAWINGS

These and other objects of the present invention will become apparent, particularly when taken in light of the following illustrations wherein:

FIG. 1 is a perspective view of the overall intra-oral imaging system;

FIG. 2 is a perspective view of the hand piece which incorporates the intra-oral camera with the swivel head optic;

FIG. 3 is a perspective view of the hand piece of the intra-oral camera with swivel head optic with the housing removed;

FIG. 4 is a perspective view of the hand piece of the intra-oral camera with swivel head optic illustrating the internal arrangement of its elements; and

FIG. 5 is an end view of the swivel head optic portion of the intra-oral cameral illustrating its swivel or pivoting capability.

### DETAILED DESCRIPTION OF THE INVENTION

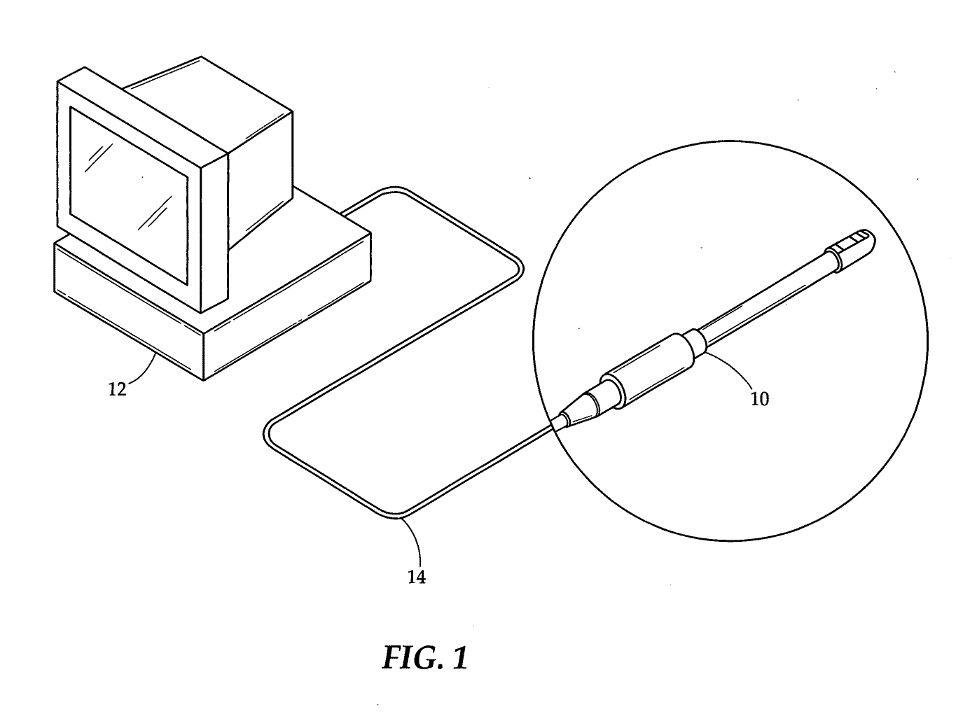

FIG. 1 is a perspective view of an intra-oral dental video imaging system which includes a hand piece 10 which incorporates an intra-oral camera as more fully discussed hereafter. Hand piece 10 is in communication with a central processing unit 12 by means of an umbilical cord 14. The central processing unit 12 is equipped with the necessary software, memory, and video display so as to allow the storage of the images transmitted by the intra-oral camera within the hand piece 10, the viewing of the images, and the manipulation and transmittal of the images.

The umbilical cord 14 is of sufficient length to allow easy manipulation of the hand piece 10 within the oral cavity and allow transmission of the images to the central processing unit 12. The umbilical cord 14 further contains a quick disconnect which allows the hand piece to be transported and used between different operatories within the dental office.

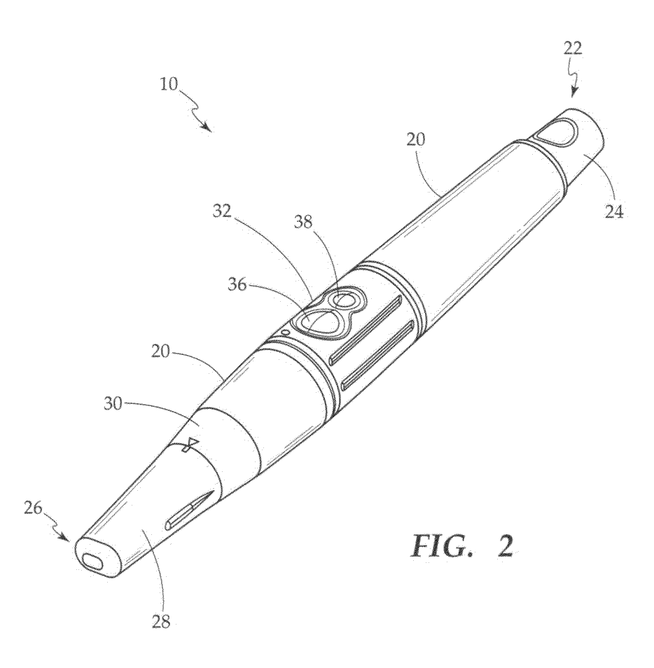

FIG. 2 is a perspective view of the hand piece 10 of the present invention. Hand piece 10 is generally longitudinally tubular in shape having an outer housing 20. A first end 22 of housing 20 is formed with a quick disconnect cable grip 24 for securing umbilical cord 14 to hand piece 10. A second end 26 of the housing 20 is formed with a rotating optic housing 28 which rotatable optic housing 28 secures a light source and an imaging prism for viewing the oral cavity. Rotating optic housing member 28 is also formed with a neutral position indicator 30 in the form of two alignable indicia which quickly and easily identifies to the user the rotational positioning of the imaging prism and camera as more fully discussed hereafter. A multi-button control panel 32 is positioned on housing 20 approximately midway between the first end 22 and the second end 26.

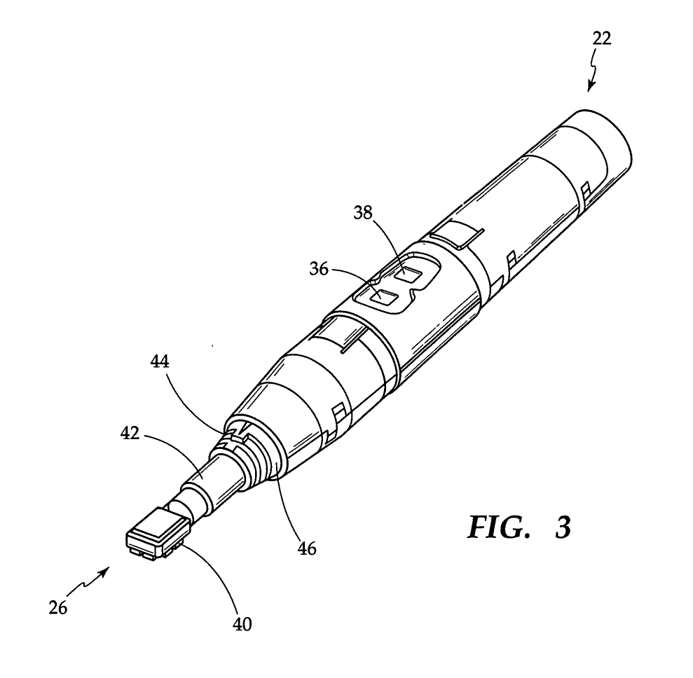

FIG. 3 is a perspective view of the hand piece 10 with housing member 20 removed. The control panel 32 located midway between first end 22 and second end 26, in the embodiment illustrated, utilizes two tactile button switches 36 and 38 to control the imaging obtained with the intra-oral camera. At the second end 26 of the hand piece, there is located an LED illumination board 40 which is in communication with the optic members 42, both of which are located within the rotatable optic housing 28, which is retained on the hand piece by a snap ring 44 which incorporates a rotation stop 46 to limit the rotation of the optic head piece to a nominal arc of 160 degrees to the right and/or left of dead center.

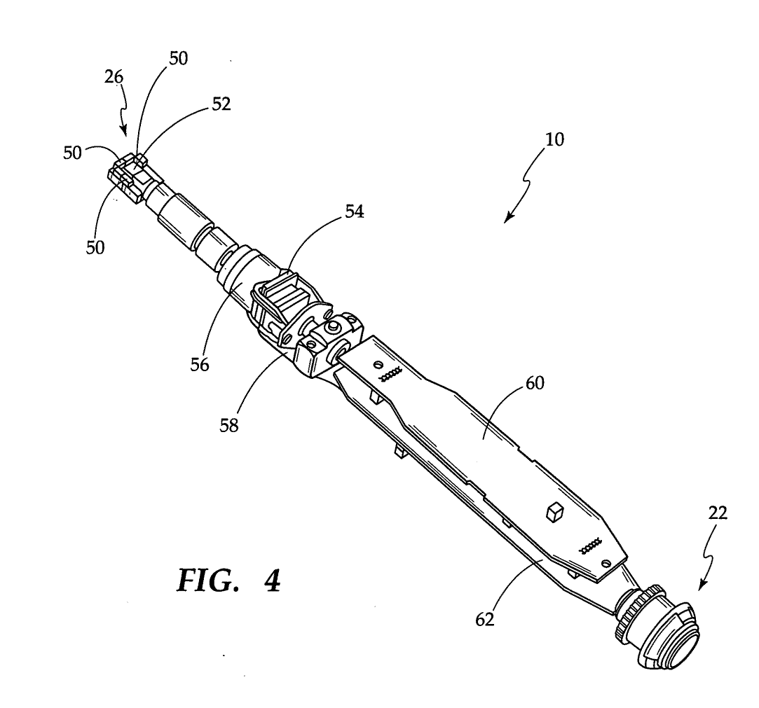

FIG. 4 is a perspective view of the hand piece 10 from the underside. Second end 26 of hand piece 10 is formed with an LED illumination board 40 which in the embodiment illustrated has six white LED's 50 arranged in a U-shaped configuration partially surrounding an imaging prism 52. The LED illumination board 40 illuminates the desired portion of the oral cavity, the reflected light being captured by the imaging prism 52 and transmitted to a charged coupled device (CCD) 54 positioned within a CCD housing 56. The CCD 54 is in communication via a flex circuit 58 with a camera board 60 mounted proximate the first end 22 of hand piece 10, together with an interface board 62 which is in communication with umbilical cord 14.

In actual operation, with the hand piece 10 secured to the umbilical cord 14 which in turn is secured to the central processing unit 12, the dentist or dental technician would predetermine the area of the oral cavity to be imaged. The dentist or dental technician would then swivel the rotatable optic housing 28 to the desired angular orientation to view the desired portion of the oral cavity. The dentist or dental technician would then insert the second end 26 of the hand piece which contains the rotatable optic housing 28 into the oral cavity with the rotatable optic housing 28 at a desired angular position. The dentist or dental technician would then manipulate the functions of hand piece 10 with the tactile switch buttons 36 and 38 located midway between first end 22 and second end 26. Tactile switch buttons 36 and 38 would activate the LED illumination board 40 and cause the reflected image to be transmitted to the CCD 54 and thence to the camera board 60, interface board 62 and the central processing unit 12. During the entire imaging process, the dentist or dental technician is viewing the imaged oral cavity on the monitor associated with central processing unit 12.

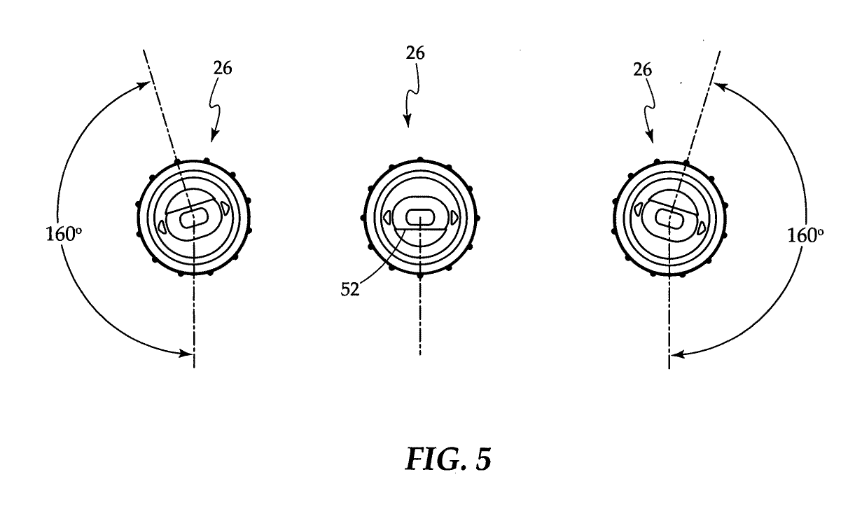

FIG. 5 is an end view of second end 26 of hand piece 10. The central illustration of FIG. 5 illustrates the imaging prism 52 in a bottom dead center position for the viewing of an object (OB). The right hand image of FIG. 5 illustrates the imaging prism 52 and the rotating optic housing 28 rotated from bottom dead center in a nominal arc of 160 degrees to the right and the left hand figure of FIG. 5 illustrates the imaging prism 50 rotated from bottom dead center in a nominal arc 160 degrees to the left. The rotatable optic housing 28 allows the dentist or dental technician to insert the second end 22 of hand piece 10 containing the rotatable optic housing 28 into the oral cavity with the imaging prism 50 at a preselected angle of orientation. In this configuration, the dentist or dental technician can image or view the desired portion of the left or right lower jaw or left or right upper jaw without having to twist and/or contort his fingers, hand or wrist to obtain such an image with an intra-oral camera which did not offer a rotatable optic housing.

Therefore, while the present invention has been disclosed with respect to the preferred embodiments thereof, it will be recognized by those of ordinary skill in the art that various changes and modifications can be made without departing from the spirit and scope of the invention. It is therefore manifestly intended that the invention be limited only by the claims and the equivalence thereof.

What is claimed is:

1. A dental video camera for examining an oral cavity, which comprises:
a housing member having a handle portion connected to a monitor and a central processing unit, said handle portion having a plurality of tactile control means secured thereto;
an optic member including a light source mounted for rotation on said housing member, said optic member including a window for receiving reflected light from said light source and directing said reflected light to a light sensing device and thence a monitor for viewing.

2. The dental video camera as defined in claim 1 wherein said light sensing device is a charged coupled device.

3. The dental video camera as defined in claim 1 wherein said optic member is rotatable to either side of a longitudinal axis of said handle member.

4. The dental video camera as defined in claim 3 and further including indicia's on said handle member and said optic member indicative of a neutral position therebetween.

5. The dental video camera as defined in claim 3 wherein said optic member is rotatable to 160 degree on either side of said neutral position.

6. The dental video camera as defined in claim 1 wherein said light source is comprised of a plurality of light emitting diodes.

7. The dental video camera as defined in claim 6 wherein said light emitting diodes have a light level of a least 70 foot candles.
